(19) United States
(12) Patent Application Publication
(10) Pub. No.: US 2025/0375625 A1
(43) Pub. Date: Dec. 11, 2025

(54) DETECTING ULTRASOUND BEAMS USING ULTRASOUND IMAGING

(71) Applicant: ACOUSTIIC INC., Bellevue, WA (US)

(72) Inventors: Holly Lay, Seattle, WA (US); Paul Reynolds, Renton, WA (US)

(73) Assignee: ACOUSTIIC INC., Bellevue, WA (US)

(21) Appl. No.: 18/740,337

(22) Filed: Jun. 11, 2024

(51) Int. Cl. A61N 7/00; A61B 8/00; G01S 15/86; G01S 15/89

(52) U.S. Cl. CPC A61N 7/00; A61B 8/4477; A61B 8/4488; A61B 8/4494; G01S 15/86; G01S 15/8927; A61B 8/4461

(57) ABSTRACT

Systems and techniques are provided for detecting therapeutic ultrasound beams using ultrasound imaging. A first transducer array includes transducer elements and is configured to generate an acoustic beam for therapy. A second transducer array and a third transducer array are arranged to image a location of a target at which the acoustic beam for therapy is directed. The second transducer array is arranged at an angle to the third transducer array such that azimuthal data of the second transducer is used to locate the elevational direction of the third transducer array. A computing and imaging device is connected to the first transducer array, the second transducer array, and the third transducer array.

### BACKGROUND

An ultrasound system may be used for focused ultrasound (FUS) therapy. An acoustic beam output by a transducer array of the ultrasound system may be controlled so that its area of effect is targeted to specific areas. The location, breadth, and amplitude of the acoustic beam may be tracked. This may be done to improve both the safety and effectiveness of FUS. An ultrasound system used for FUS therapy may use a 2D transducer array for 3D volumetric imaging and to output an acoustic beam suitable for FUS therapy, ensuring co-registration. Ultrasound systems used for FUS therapy may use one device with a transducer array to generate the acoustic beam for FUS therapy and any number of other, separate, non-ultrasound devices, for imaging. This may make it more difficult to locate targets for the application of the acoustic beam for FUS therapy, to monitor the FUS therapy while it is in progress, and to assess the outcome of the therapy. Ultrasound systems that use separate devices for therapy and imaging may use a focused bowl device with a central hole containing a lower power imaging transducer that may be able generate adequate images along a central axis but not off that axis, along with separate non-ultrasound imaging devices. Transducer arrays used for imaging may have a single or limited number of rows in elevation but be multi-element in azimuth. This may result in increased accuracy of the acoustic beam along the azimuth but variable performance in elevation and depth due to the shape of the acoustic beam and the inability to steer the acoustic beam with a high degree of accuracy.

### BRIEF DESCRIPTION OF THE DRAWINGS

The accompanying drawings, which are included to provide a further understanding of the disclosed subject matter, are incorporated in and constitute a part of this specification. The drawings also illustrate implementations of the disclosed subject matter and together with the detailed description serve to explain the principles of implementations of the disclosed subject matter. No attempt is made to show structural details in more detail than may be necessary for a fundamental understanding of the disclosed subject matter and various ways in which it may be practiced.

FIG. 1 shows an example system for detecting ultrasound beams using ultrasound imaging according to an implementation of the disclosed subject matter.

FIG. 2 shows an example arrangement for detecting ultrasound beams using ultrasound imaging according to an implementation of the disclosed subject matter.

FIG. 3 shows an example arrangement for detecting ultrasound beams using ultrasound imaging according to an implementation of the disclosed subject matter.

FIG. 4 shows an example procedure for detecting ultrasound beams using ultrasound imaging according to an implementation of the disclosed subject matter.

FIG. 5 shows an example arrangement for detecting ultrasound beams using ultrasound imaging according to an implementation of the disclosed subject matter.

FIG. 6 shows an example procedure for detecting ultrasound beams using ultrasound imaging according to an implementation of the disclosed subject matter.

FIG. 7 shows an example arrangement for detecting ultrasound beams using ultrasound imaging according to an implementation of the disclosed subject matter.

FIG. 8 shows an example procedure for detecting ultrasound beams using ultrasound imaging according to an implementation of the disclosed subject matter.

FIG. 9 shows a computer according to an implementation of the disclosed subject matter.

FIG. 10 shows a network configuration according to an implementation of the disclosed subject matter.

### DETAILED DESCRIPTION

An ultrasound system may use one or more imaging transducer arrays in conjunction with a therapy transducer array. This may improve performance over an ultrasound system that uses a single imaging transducer array, for example, when performing FUS therapy. For example, an ultrasound system may use two 1D imaging transducer arrays. A single 1D transducer array may include a single 1×N row of transducer elements. The two 1D imaging transducer arrays may be placed at known locations relative to a device with a transducer array that generates an acoustic beam for therapy. The two 1D imaging transducer arrays may be arranged at an angle to one another, for example, at 90 degrees, and oriented so their fields of view overlap in a region of interest, for example, a region that includes the target for the acoustic beam from a transducer array used for therapy. The imaging transducer(s) have a known location and orientation with respect to the therapy transducer. The ultrasound system may use any number of transducer arrays for imaging. The imaging transducer arrays may or may not fully encompass the therapy transducer array used to generate an acoustic beam for therapy and may or may not be interspersed within the aperture of the therapy transducer array. The imaging transducer arrays may operate in pitch catch or pulse echo modes or may use the pulses of the acoustic beam generated by the therapy transducer array or pulses from other imaging transducer arrays as the source of the reflected ultrasound used for imaging. Imaging transducer arrays may be selected to match the elevation foci of the imaging transducer arrays to the target depth of the acoustic beam for therapy, or known landmarks, such as anatomic landmarks, in the target, or to other clinical depth characteristics. Imaging transducer arrays may be swappable to match multiple applications, including clinical applications, and desired specifications of the ultrasound system.

The imaging transducer arrays of the ultrasound system may use any suitable imaging mode type, such as, for example, b-mode, harmonic, shear wave, and plane wave imaging.

An ultrasound system with multiple imaging transducer arrays and a therapy transducer array may be used to, for example, locate a target within a patient, using the imaging transducer arrays, apply therapy treatment using an acoustic beam generated by the therapy transducer array, and use the ultrasound time-of-flight data as determined by the multiple imaging transducer arrays to generate improved beam steering data, including amplitudes and delay times. The beam steering data may be used to adjust the acoustic beam generated by the therapy transducer array.

An ultrasound system with multiple imaging transducer arrays and a therapy transducer array may be used to locate a response to applied ultrasound pulses from the acoustic beam generated by the therapy transducer array in 3D space and relative to other markers. The response may be, for example, the occurrence of cavitation. Data from the imaging transducer arrays may be used to estimate characteristics, such as an overall size, of the response.

An ultrasound system with multiple imaging transducer arrays and a therapy transducer array may be used to detect physical changes in a target, such as tissue, due to the acoustic beam generated by the therapy transducer array or other factors, such as movement of the target. For example, if the target is within a patient, breathing or the patient's cardiac cycle may result in the target moving.

The output data from the imaging transducer arrays of the ultrasound system based on the detection of reflected ultrasound waves may be any of: raw ultrasound data that is either pre- or post-beamformed, gray scale ultrasound images, post-processed image data, or probability maps of beam locations. The output data from the imaging transducer arrays may be communicated directly to the ultrasound system via a joint control system or may be communicated electronically between multiple control systems using any suitable data formats, such as, for example, DICOM. The output data from the imaging transducer arrays may be used as part of a feedback loop within the ultrasound system, either in real time or at a suitable high frequency, for example, in between or just before pulses of the acoustic beam from the therapy transducer array. The ultrasound system may also include synchronization control between all transducers of the therapy transducer array and the imaging transducer arrays to allow simultaneous firing or offset firing depending on imaging/monitoring mode to remove artefacts due to acoustic and/or electronic interference.

The ultrasound system may be used in conjunction with other imaging devices, such as magnetic resonance (MR) or computed tomography (CT) imagers. These images may be taken concurrently with ultrasound imaging or at a different time and used as input in any calculations. The beam steering data, which may include, for example, time-of-flight data, from the imaging transducer arrays may be enhanced using image fusion with images generated by the other imaging devices or may be cross-referenced with the images generated by the other imaging devices to confirm results. The image fusion may also be used to improve localization of landmarks around the target.

The imaging transducer arrays may be moved, having their locations or orientations changed, in order to improve imaging of particular regions of interest or to improve locating signals. Moving the imaging transducer arrays by small amounts may increase localization accuracy or volume estimation. Any number of the imaging transducer arrays may be moved at any given time. Movement of the imaging transducer arrays may be manual, based on pre-programmed algorithms using computer control, may be based on qualitative or quantitative data from the imaging transducer arrays, or may be based on a machine learning system trained on data from previous uses of the ultrasound system. Computer controlled motion may be performed using mechanical or electronic motion stages or robotic control arms.

The imaging transducer arrays may be of any type, such as, for example, linear, curved, phased, microconvex, single row, 1.25D multi row, 1.5D multi row, 1.75D multi row, and 2D matrix with a field of view capable of imaging the target. More advanced elevational steering or control may allow for better performance of the imaging transducer arrays.

The imaging transducer arrays may operate in a pulse echo manner, where the same transducer array both sends an ultrasound pulse and receives the reflected ultrasound resulting from the ultrasound pulse, or a pitch catch manner, where one of the imaging transducer arrays sends the ultrasound pulse and another imaging transducer array receives the reflected ultrasound resulting from the ultrasound pules. The imaging transducer arrays may use matrix capture by transmitting ultrasound on a single transducer element or group of transducer elements at one time and receiving reflected ultrasound on all transducer elements and repeating this until all transducer elements or groups of transducer elements have transmitted, plane wave capture by transmitting ultrasound in a single broad wave at multiple angles and then receiving the reflected ultrasound, or any other form of ultrasound imaging such as, for example, doppler imaging or harmonic imaging.

For an ultrasound system that uses a single imaging transducer array, or more than one imaging transducer array with no angle between the imaging transducer arrays, when any clearly distinguished target, such as, for example, an anatomical marker, a response to therapy waves such as cavitation, or an implanted source, is visible to the imaging transducer arrays, the target may be accurately located in azimuth but not in elevation. Placing imaging transducer arrays at angles to one another may allow the azimuthal data from one imaging transducer array to be used to locate the elevational direction of another imaging transducer array, and vice versa. The angle used may be, for example, 90 degrees, though other angles, for example, greater than 0 degrees and less than 180 degrees, may be used when there is more than one imaging transducer array or a single imaging transducer array with transducer elements grouped into an angled or curved shape.

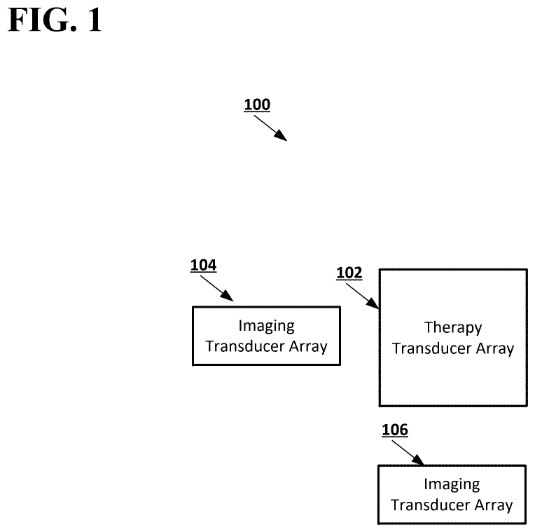

FIG. 1 shows an example system for detecting ultrasound beams using ultrasound imaging according to an implementation of the disclosed subject matter. An ultrasound system 100 may include therapy transducer array 102 and imaging transducer arrays 104 and 106. The therapy transducer array 102 may be an M×N array of any suitable size, with any suitable number of transducer elements, that may generate an acoustic beam that may be used for therapy. The imaging transducer arrays 104 and 106 may be, for example, 1-dimensional transducer arrays with 1×N arrays of transducer elements, linear, curved, phased, microconvex, single row, 1.25D multi row, 1.5D multi row, 1.75D multi row, and 2D matrix with a field of view capable of imaging the target. The imaging transducer arrays 104 and 106 may be arranged so that they may image a region of interest that includes a target of an acoustic beam generated by the therapy transducer array 102. The fields of view of the imaging transducer arrays 104 and 106 may overlap. The ultrasound system 100 may include any number, greater than one, of imaging transducer arrays such as the imaging transducer arrays 104 and 106.

The imaging transducer arrays 104 and 106 may operate in pitch catch or pulse echo modes or may use the pulses of the acoustic beam generated by the therapy transducer array 102 or pulses from other imaging transducer arrays. The imaging transducer arrays 104 and 106 may be selected to match elevation foci of the imaging transducer arrays 104 and 106 to the target depth of the acoustic beam for therapy generated by therapy transducer array 102, or known landmarks, such as anatomic landmarks, in the target, or to other clinical depth characteristics. The imaging transducer arrays 104 and 106 may be swappable to match multiple applications, including clinical applications, and desired specifications of the ultrasound system 100.

The imaging transducer arrays 104 and 106 of the ultrasound system 100 may use any suitable imaging mode type, such as, for example, b-mode, harmonic, shear wave, and plane wave imaging.

The ultrasound system 100, including the imaging transducer arrays 104 and 106 and a therapy transducer array 102 may be used to locate a target, for example, within a patient, using the imaging transducer arrays 104 and 106, apply therapy treatment using an acoustic beam generated by the therapy transducer array 102, and use the ultrasound time-of-flight data as determined by the imaging transducer arrays 104 and 106 to generate improved beam steering data, including amplitudes and delay times. The beam steering data may be used to adjust the acoustic beam generated by the therapy transducer array 102.

The ultrasound system 100 may be used to locate a response to applied ultrasound pulses from the acoustic beam generated by the therapy transducer array 102 in 3D space and relative to other markers. The response may be, for example, the occurrence of cavitation. Data from the imaging transducer arrays 104 and 106 may be used to estimate characteristics, such as an overall size, of the response.

The ultrasound system 100 may be used to detect physical changes in a target, such as tissue, due to the acoustic beam generated by the therapy transducer array 102 or other factors, such as movement of the target. For example, if the target is within a patient, breathing or the patient's cardiac cycle may result in the target moving.

The output data from the imaging transducer arrays 104 and 106 of the ultrasound system 100 based on the detection of reflected ultrasound waves may be any of: raw ultrasound data that is either pre- or post-beamformed, gray scale ultrasound images, post-processed image data, or probability maps of beam locations. The output data from the imaging transducer arrays 104 and 106 may be communicated directly to the ultrasound system 100 via a joint control system or may be communicated electronically between multiple control systems using any suitable data formats, such as, for example, DICOM. The output data from the imaging transducer arrays 104 and 106 may be used as part of a feedback loop within the ultrasound system 100, either in real time or at a suitable high frequency, for example, in between or just before pulses of the acoustic beam from the therapy transducer array 102. The ultrasound system 100 may also include synchronization control between all transducers of the therapy transducer array 102 and the imaging transducer arrays 104 and 106 to allow simultaneous firing or offset firing depending on imaging/monitoring mode to remove artefacts due to acoustic and/or electronic interference.

The ultrasound system 100 may be used in conjunction with other imaging devices, or images from other devices, such as magnetic resonance (MR) or computed tomography (CT) imagers. The beam steering data, which may include, for example, time-of-flight data, from the imaging transducer arrays 104 and 106 may be enhanced using image fusion with images generated by the other imaging devices, or may be cross-referenced with the images generated by the other imaging devices to confirm results. The image fusion may also be used to improve localization of landmarks around the target.

The imaging transducer arrays 104 and 106 may be moved, having their locations or orientations changed, in order to improve imaging of particular regions of interest or to improve locating signals. Moving the imaging transducer arrays 104 and 106 by small amounts may increase localization accuracy or volume estimation. Any of the imaging transducer arrays 104 and 106 may be moved at any given time. Movement of the imaging transducer arrays 104 and 106 may be manual, based on pre-programmed algorithms using computer control, may be based on qualitative or quantitative data from the imaging transducer arrays, or may be based on a machine learning system trained on data from previous uses of the ultrasound system. Computer controlled motion may be performed using mechanical or electronic motion stages or robotic control arms.

The imaging transducer arrays 104 and 106 may be of any type, such as, for example, linear, curved, phased, microconvex, single row, 1.25D multi row, 1.5D multi row, 1.75D multi row, and 2D matrix with a field of view capable of imaging the target. More advanced elevational steering or control may allow for better performance of the imaging transducer arrays.

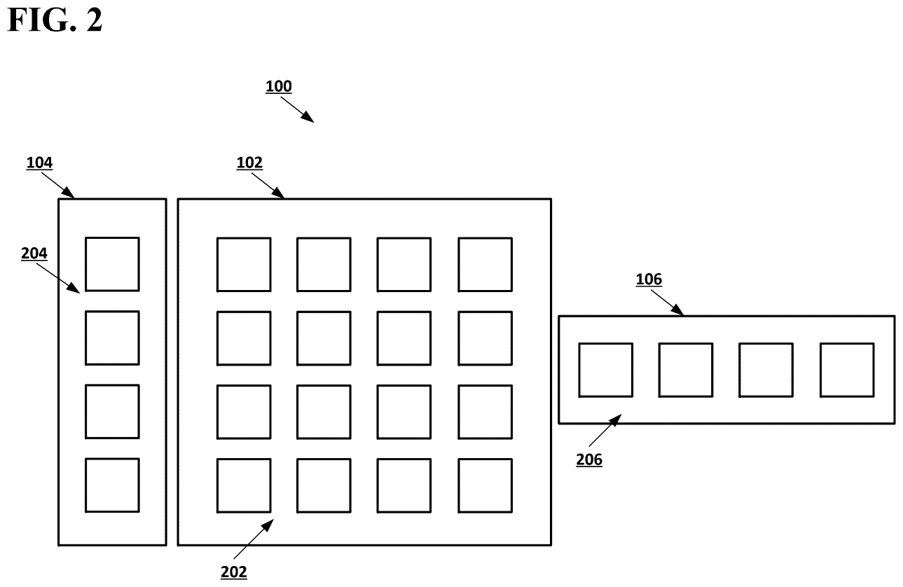

FIG. 2 shows an example system for detecting ultrasound beams using ultrasound imaging according to an implementation of the disclosed subject matter. The therapy transducer array 102 may be an array of transducer elements 202 arranged in a matrix. The transducer elements 202 may be ultrasonic transducers. The imaging transducer arrays 104 and 106 may be, for example, arrays of transducer elements 204 and 206 arranged linearly, or may be in any other suitable form, including for example, linear, curved, phased, microconvex, single row, 1.25D multi row, 1.5D multi row, 1.75D multi row, and 2D matrix with a field of view capable of imaging the target. The transducer elements 204 and 206 may be ultrasonic transducers of the same type as or different type from the transducer elements 202. The imaging transducer arrays 104 and 106 may be placed at angles to one another, which may allow the azimuthal data from one imaging transducer arrays 104 and 106 to be used to locate the elevational direction of the other of the imaging transducer arrays 104 and 106, and vice versa. The angle used may be, for example, 90 degrees, though other angles may be used.

In some implementations, imaging transducer arrays, such as the imaging transducer arrays 104 and 106, may be arranged across the face of the therapy transducer array 102. For example, imaging transducer arrays such as the imaging transducer arrays 104 and 106 may be made from thin membrane Polyvinylidene Fluoride (PVDF) and may be fully sampled or row or column transducer arrays that may be placed within the aperture of the therapy transducer array 102 at angels to one another.

In some implementations, imaging transducer arrays, such as the imaging transducer arrays 104 and 106, may be implemented using space on the therapy transducer array 102. For example, single rows and/or columns of transducer elements of the therapy transducer array 102 may be used as imaging transducer arrays, such as the imaging transducer arrays 104 and 106.

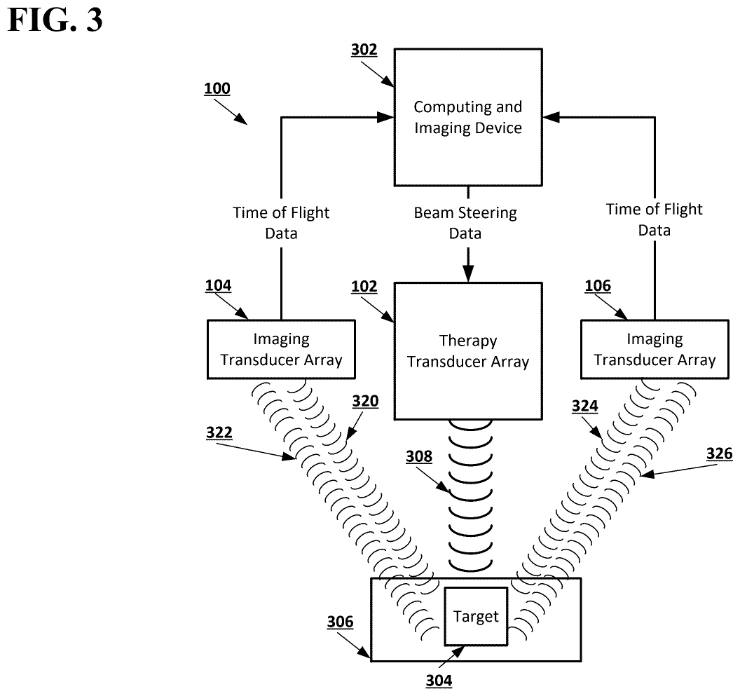

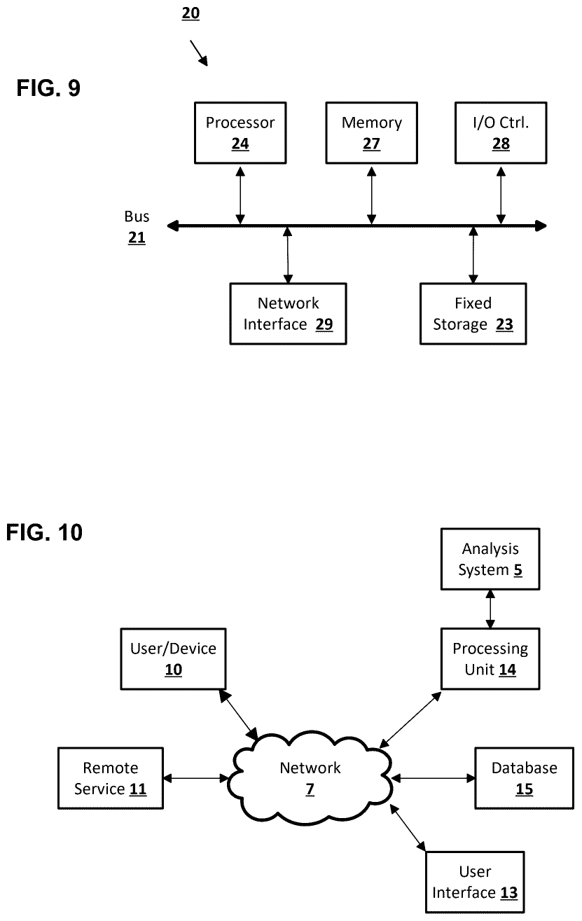

FIG. 3 shows an example arrangement for detecting ultrasound beams using ultrasound imaging according to an implementation of the disclosed subject matter. The ultrasound system 100 may include a computing and imaging device 302. The therapy transducer array 102 and the imaging transducer arrays 104 and 106 may be connected to the computing and imaging device 302 through any suitable wired and/or wireless connections. The computing and imaging device 102 may include any suitable computing hardware, running any suitable software, and any other suitable electronics to operate the ultrasound system 100, including supplying power and control signals to transducer elements of the therapy transducer array 102 and the imaging transducer arrays 104 and 106, receiving signals from the transducer elements of the therapy transducer array 102 and the imaging transducer arrays 104 and 106, performing any suitable computation to generate images from the signals received from the transducer elements of the therapy transducer array 102 and the imaging transducer arrays 104 and 106, and displaying generated images, for example, on a display directly connection to the computing and imaging device 302, or otherwise sending the generated images to a device, for example, a tablet or phone, that can display the generated images. The computing and imaging device 302 may have any suitable interface to allow a user to control the ultrasound system 100. The computing and imaging device 102 may be or include a computer 20 as shown in in FIG. 9. The computing and imaging device 302 may also include any suitable electric and electronic components for delivering power to the therapy transducer array 102 and the imaging transducer arrays 104 and 106.

The ultrasound system 100, including the imaging transducer arrays 104 and 106 and a therapy transducer array 102 may be used to locate a target 304, for example, within a region of interest of a volume 306, such as a patient, using the imaging transducer arrays 104 and 106, and apply therapy treatment using an acoustic beam 308 generated by the therapy transducer array 102. The computing and imaging device 302 may use the ultrasound time-of-flight data as determined by the imaging transducer arrays 104 and 106 to generate improved beam steering data, including amplitudes and delay times. The beam steering data may be used by the computing and imaging device 302 to adjust the acoustic beam generated by the therapy transducer array 102. The ultrasound time of time-of-flight may be determined based on the transmission of acoustic beams 320 and 324 and the detection of reflected ultrasound 322 and 326 by the imaging transducer arrays 104 and 106. The reflected ultrasound 322 and 326 may be ultrasound of the acoustic beams 320 and 324 as reflected by the volume 306 and the target 304. The reflected ultrasound 322 and 326 may also result from reflection of the acoustic beam 308 generated by the therapy transducer array 102.

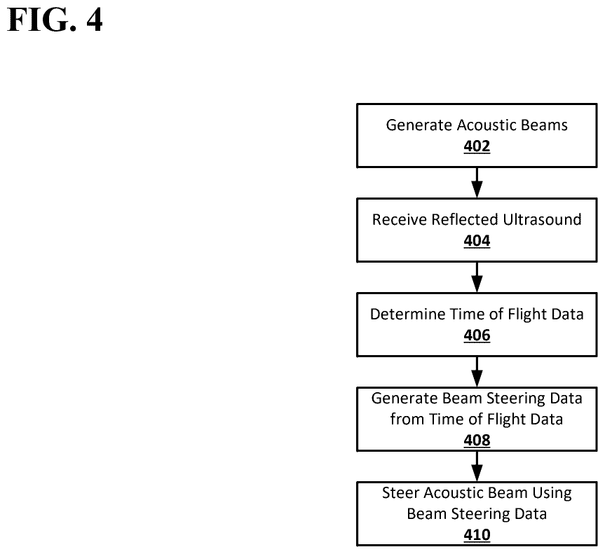

FIG. 4 shows an example procedure for detecting ultrasound beams using ultrasound imaging according to an implementation of the disclosed subject matter. At 402, acoustic beams may be generated. For example, the imaging transducer arrays of an ultrasound system, such as the imaging transducer arrays 104 and 106 of the ultrasound system 100, and/or therapy transducer arrays, such as the therapy transducer array 102, may be used to generate acoustic beams, such as the acoustic beams 320, 324 and 308. The acoustic beams 308, 320 and 324 may be directed towards the area in which a target, such as the target 304, is believed to be located within a volume, for example, the volume 306, which may be, for example, a patient.

At 404, reflected ultrasound may be received. For example, any of the acoustic beams 308, 320 and 324 may reflect off of material of the volume 306 and the target 304, resulting in reflected ultrasound 322 and 326 that may be received at the transducer elements of the imaging transducer arrays 104 and 106.

At 406, time-of-flight data may be determined. For example, the imaging transducer arrays 104 and 106 may determine the time-of-flight or amplitude of any of the acoustic beams 308, 320 and 324 to the target 304 based on, for example, the time between the generation of the acoustic beams 308, 320 and 324 and the receiving of the reflected ultrasound 322 and 326.

At 408, beam steering data may be generated from the time-of-flight or amplitude data. For example, the computing and imaging device 302 may use the time-of-flight or amplitude data to generate improved beam steering data for the acoustic beam 308. The improved beam steering data may, for example, better direct the acoustic beam 308 at the target 304.

At 410, an acoustic beam may be steered using the beam steering data. For example, the computing and imaging device 302 may use the beam steering data to steer the acoustic beam 308 generated by the therapy transducer array 102. The beam may be steered, for example, to be better directed at the target 304.

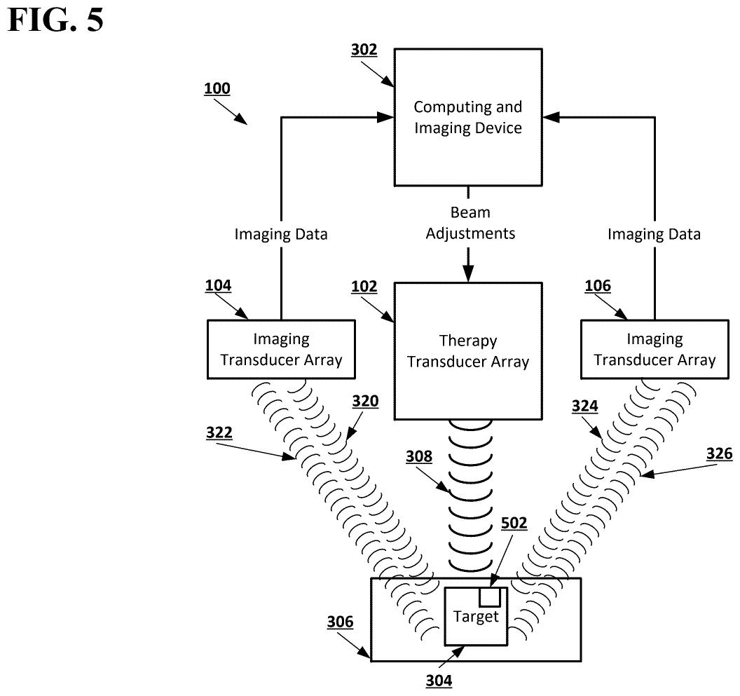

FIG. 5 shows an example arrangement for detecting ultrasound beams using ultrasound imaging according to an implementation of the disclosed subject matter. The ultrasound system 100 may locate a response 502 to applied ultrasound pulses from the acoustic beam 308 generated by the therapy transducer array 102 in 3D space and relative to other markers. The response 502 may be, for example, the occurrence of cavitation in the target 304. The imaging transducer arrays 104 and 106 may generate imaging data from the reflected ultrasound 322 and 326. The imaging data may be sent from the imaging transducer arrays 104 and 106 to the computing and imaging device 302 which may use the imaging data to estimate characteristics, such as an overall size, of the response 502.

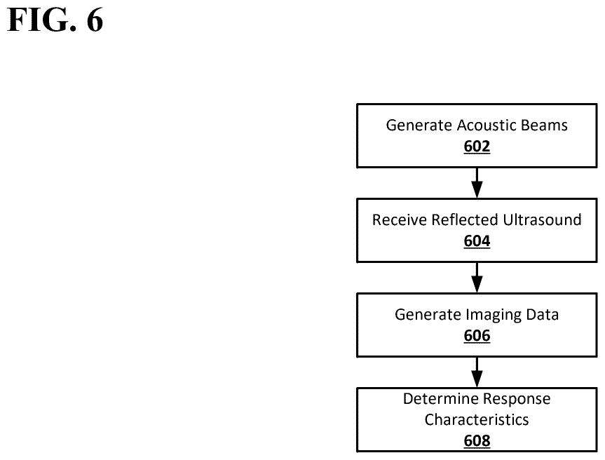

FIG. 6 shows an example procedure for detecting ultrasound beams using ultrasound imaging according to an implementation of the disclosed subject matter. At 602, acoustic beams may be generated. For example, the imaging transducer arrays of an ultrasound system, such as the imaging transducer arrays 104 and 106 of the ultrasound system 100, and/or therapy transducer arrays, such as the therapy transducer array 102, may be used to generate acoustic beams, such as the acoustic beams 320, 324 and 308. The acoustic beams 308, 320 and 324 may be directed towards the area in which a response, such as the response 502, which may be cavitation, of the target 304 to the acoustic beam 308 is believed to be located within the volume 306, which may be, for example, a patient.

At 604, reflected ultrasound may be received. For example, any of the acoustic beams 308, 320 and 324 may reflect off of material of the volume 306 and the target 304, including the area of the response 502, resulting in the reflected ultrasound 322 and 326 that may be received at the transducer elements of the imaging transducer arrays 104 and 106.

At 606, imaging data may be generated. For example, the imaging transducer arrays 104 and 106 may generate imaging data based off the reflected ultrasound 322 and 326. The reflected ultrasound 322 and 326 may be received at transducer elements of the imaging transducer arrays 104 and 106 which may operate in a receiving mode and may generate signals based on the reflected ultrasound 322 and 326. The generated signals may be the imaging data. The imaging data may be data that may be used to generate images of the area within the fields of view of the imaging transducer arrays 104 and 106, which may, for example, include the target 304.

At 608, response characteristics may be determined. For example, the imaging data may be sent from the imaging transducer arrays 104 and 10 to the computing and imaging device 302. The computing and imaging device 302 may use the imaging data to determine characteristics, such as size, for the response 502. The response characteristics may be used in any suitable manner, including, for example, to adjust any suitable properties of the acoustic beam 308.

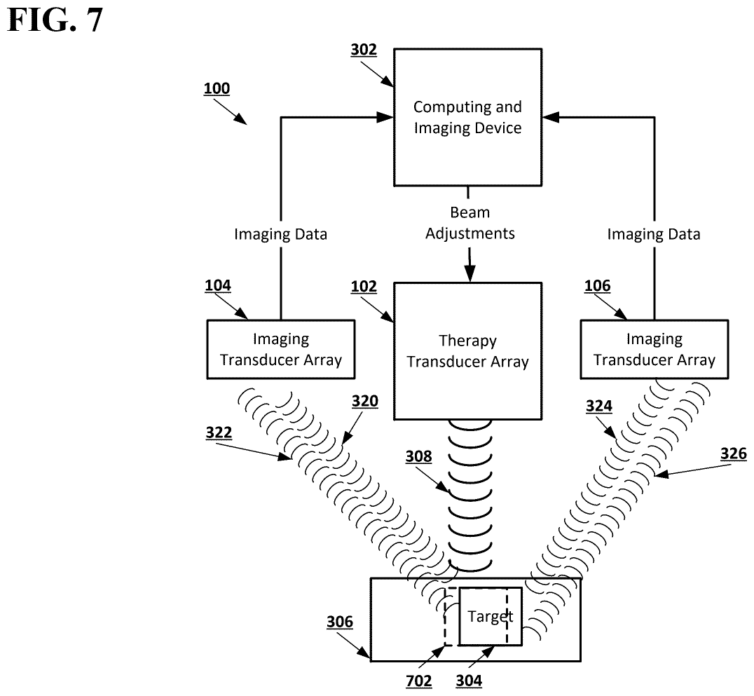

FIG. 7 shows an example arrangement for detecting ultrasound beams using ultrasound imaging according to an implementation of the disclosed subject matter. The ultrasound system 100 may use the imaging transducer arrays 104 and 106 to generate image data that may be used to detect physical changes in the target 304, such as tissue, due to the acoustic beam 308 generated by the therapy transducer array 102 or other factors, such as movement of the target 304. For example, if the target 304 is within a patient, breathing or the patient's cardiac cycle may result in the target 304 moving, for example, from the position 702 to a current position. The detection of the physical changes in the target 304 may be used, for example, adjust properties of the acoustic beam 308.

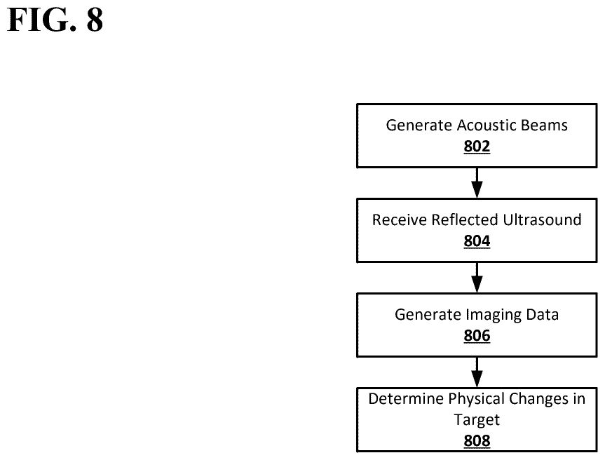

FIG. 8 shows an example procedure for detecting ultrasound beams using ultrasound imaging according to an implementation of the disclosed subject matter. At 802, acoustic beams may be generated using imaging transducer arrays. For example, the imaging transducer arrays of an ultrasound system, such as the imaging transducer arrays 104 and 106 of the ultrasound system 100, may be used to generate acoustic beams, such as the acoustic beams 320 and 324. The acoustic beams 320 and 324 may be directed towards the area in which a response, such as the response 502, which may be cavitation, of the target 304 to the acoustic beam 308 is believed to be located the volume 306, which may be, for example, a patient.

At 804, reflected ultrasound may be received. For example, any of the acoustic beams 308, 320 and 324 may reflect off of material of the volume 306 and the target 304, including the area of the response 502, resulting in the reflected ultrasound 322 and 326 that may be received at the transducer elements of the imaging transducer arrays 104 and 106.

At 806, imaging data may be generated. For example, the imaging transducer arrays 104 and 106 may generate imaging data based on the reflected ultrasound 322 and 326. The reflected ultrasound 322 and 326 may be received at transducer elements of the imaging transducer arrays 104 and 106 which may operate in a receiving mode and may generate signals based on the reflected ultrasound 322 and 326. The generated signals may be the imaging data. The imaging data may be data that may be used to generate images of the area within the fields of view of the imaging transducer arrays 104 and 106, which may, for example, include the target 304.

At 808, physical changes in the target may be determined. For example, the imaging data may be sent from the imaging transducer arrays 104 and 10 to the computing and imaging device 302. The computing and imaging device 302 may use the imaging data to determine any physical changes in the target 304, such as, for example, a change in the location of the target 304, or a change in the properties of the material, for example, tissue, of the target 304. The physical changes in the target 304 may be used in any suitable manner, including, for example, to adjust any suitable properties of the acoustic beam 308.

Implementations of the presently disclosed subject matter may be implemented in and used with a variety of component and network architectures. FIG. 9 is an example computer 10 suitable for implementations of the presently disclosed subject matter. The computer 10 includes a bus 11 which interconnects major components of the computer 10, such as a central processor 24, a memory 27 (typically RAM, but which may also include ROM, flash RAM, or the like), an input/output controller 28, a user display 22, such as a display screen via a display adapter, a user input interface 26, which may include one or more controllers and associated user input devices such as a keyboard, mouse, and the like, and may be closely coupled to the I/O controller 28, fixed storage 23, such as a hard drive, flash storage, Fibre Channel network, SAN device, SCSI device, and the like, and a removable media component 25 operative to control and receive an optical disk, flash drive, and the like.

The bus 11 allows data communication between the central processor 24 and the memory 27, which may include read-only memory (ROM) or flash memory (neither shown), and random access memory (RAM) (not shown), as previously noted. The RAM is generally the main memory into which the operating system and application programs are loaded. The ROM or flash memory can contain, among other code, the Basic Input-Output system (BIOS) which controls basic hardware operation such as the interaction with peripheral components. Applications resident with the computer 10 are generally stored on and accessed via a computer readable medium, such as a hard disk drive (e.g., fixed storage 23), an optical drive, floppy disk, or other storage medium 25.

The fixed storage 23 may be integral with the computer 10 or may be separate and accessed through other interfaces. A network interface 29 may provide a direct connection to a remote server via a telephone link, to the Internet via an internet service provider (ISP), or a direct connection to a remote server via a direct network link to the Internet via a POP (point of presence) or other technique. The network interface 29 may provide such connection using wireless techniques, including digital cellular telephone connection, Cellular Digital Packet Data (CDPD) connection, digital satellite data connection, or the like. For example, the network interface 29 may allow the computer to communicate with other computers via one or more local, wide-area, or other networks, as shown in FIG. 10.

Many other devices or components (not shown) may be connected in a similar manner (e.g., document scanners, digital cameras, and so on). Conversely, all of the components shown in FIG. 9 need not be present to practice the present disclosure. The components can be interconnected in different ways from that shown. The operation of a computer such as that shown in FIG. 9 is readily known in the art and is not discussed in detail in this application. Code to implement the present disclosure can be stored in computer-readable storage media such as one or more of the memory 27, fixed storage 23, removable media 25, or on a remote storage location.

FIG. 10 shows an example network arrangement according to an implementation of the disclosed subject matter. One or more clients 10, 11, such as local computers, smart phones, tablet computing devices, and the like may connect to other devices via one or more networks 7. The network may be a local network, wide-area network, the Internet, or any other suitable communication network or networks, and may be implemented on any suitable platform including wired and/or wireless networks. The clients may communicate with one or more servers 13 and/or databases 15. The devices may be directly accessible by the clients 10, 11, or one or more other devices may provide intermediary access such as where a server 13 provides access to resources stored in a database 15. The clients 10, 11 also may access remote platforms 17 or services provided by remote platforms 17 such as cloud computing arrangements and services. The remote platform 17 may include one or more servers 13 and/or databases 15.

More generally, various implementations of the presently disclosed subject matter may include or be implemented in the form of computer-implemented processes and apparatuses for practicing those processes. The disclosed subject matter also may be implemented in the form of a computer program product having computer program code containing instructions implemented in non-transitory and/or tangible media, such as floppy diskettes, CD-ROMs, hard drives, USB (universal serial bus) drives, or any other machine readable storage medium, wherein, when the computer program code is loaded into and executed by a computer, the computer becomes an apparatus for practicing implementations of the disclosed subject matter. Implementations also may be implemented in the form of computer program code, for example, whether stored in a storage medium, loaded into and/or executed by a computer, or transmitted over some transmission medium, such as over electrical wiring or cabling, through fiber optics, or via electromagnetic radiation, wherein when the computer program code is loaded into and executed by a computer, the computer becomes an apparatus for practicing implementations of the disclosed subject matter. When implemented on a general-purpose microprocessor, the computer program code segments configure the microprocessor to create specific logic circuits. In some configurations, a set of computer-readable instructions stored on a computer-readable storage medium may be implemented by a general-purpose processor, which may transform the general-purpose processor or a device containing the general-purpose processor into a special-purpose device configured to implement or carry out the instructions.

Implementations may use hardware that includes a processor, such as a general-purpose microprocessor, one or more Field Programmable Gate Arrays (FPGAs) and/or one or more Application Specific Integrated Circuits (ASICs) that embodies all or part of the techniques according to embodiments of the disclosed subject matter in hardware and/or firmware. The processor may be coupled to memory, such as RAM, ROM, flash memory, a hard disk or any other device capable of storing electronic information. The memory may store instructions adapted to be executed by the processor to perform the techniques according to embodiments of the disclosed subject matter.

The foregoing description, for purpose of explanation, has been described with reference to specific implementations. However, the illustrative discussions above are not intended to be exhaustive or to limit implementations of the disclosed subject matter to the precise forms disclosed. Many modifications and variations are possible in view of the above teachings. The implementations were chosen and described in order to explain the principles of implementations of the disclosed subject matter and their practical applications, to thereby enable others skilled in the art to utilize those implementations as well as various implementations with various modifications as may be suited to the particular use contemplated.

1. An ultrasound system comprising:
a first transducer array comprising transducer elements and configured to generate an acoustic beam for therapy;
a second transducer array and a third transducer array arranged to image a location of a target at which the acoustic beam for therapy is directed, wherein the second transducer array is arranged at an angle to the third transducer array such that azimuthal data of the second transducer is used to locate the elevational direction of the third transducer array; and
a computing and imaging device connected to the first transducer array, the second transducer array, and the third transducer array.

2. The system of claim 1, wherein the angle is at least 90 degrees.

3. The system of claim 1, wherein the second transducer array is linear, curved, phased, microconvex, single row, 1.25D multi row, 1.5D multi row, 1.75D multi row, or 2D matrix and wherein the third transducer array is linear, curved, phased, microconvex, single row, 1.25D multi row, 1.5D multi row, 1.75D multi row, or 2D matrix.

4. The system of claim 1, wherein the computing and imagine device uses time-of-flight data determined from the second transducer array and the third transducer array to generate beam steering data for the acoustic beam generated by the first transducer array.

5. The device of system 1, wherein the computing and imaging device uses imaging data from the second transducer array and the third transducer array to determine characteristics of a response near a location of the target to the acoustic beam generated by the first transducer array.

6. The system of claim 1, wherein the computing and imaging device uses imaging data from the second transducer array and the third transducer array to determine physical changes in the target.

7. The system of claim 1, further comprising one or more additional transducer arrays, wherein the second transducer array, the third transducer array, and the one or more additional transducer arrays are imaging transducer arrays.

8. The system of claim 7, wherein fields of view the second transducer array, the third transducer array, and the one or more additional transducer arrays overlap in a region of interest that comprises the target.

9. The system of claim 1, wherein the second transducer array and the third transducer array have elevation foci matched to a target depth of the acoustic beam generated by the first transducer array or to known landmarks of the target.

10. The system of claim 1, wherein the second transducer array and third transducer array are arranged across the face of the first transducer array.

11. The system of claim 1, further comprising one or more additional transducer arrays arranged at one or more angles to each other.

12. A method comprising:
generating, one or more acoustic beams directed at a target with one or more of a first transducer array, a second transducer array, and a third transducer array, of an ultrasound system;
receiving, at the second transducer array and the third transducer array, reflected ultrasound that results from reflections of the one or more acoustic beams;
determining time-of-flight data for at least one of the one or more acoustic beams based on the receiving of the reflected ultrasound;
generating, by a computing and imaging device of the ultrasound system, beam steering data using the time-of-flight data; and
adjusting, by a computing and imaging device and using the beam steering data, an acoustic beam generated by the first transducer array.

13. The method of claim 12, wherein the second transducer array is arranged at an angle to the third transducer array such that azimuthal data of the second transducer is used to locate the elevational direction of the third transducer array, and the azimuthal data of the third transducer is used to locate the elevational direction of the second transducer array.

14. The method of claim 12, wherein fields of view the second transducer array and the third transducer array overlap in a region of interest that comprises the target.

15. The method of claim 12, wherein generating, by a computing and imaging device of the ultrasound system, beam steering data further comprises performing image fusion with images generated by one or more other imaging devices.

16. The method of claim 12, further comprising moving the second transducer array and the third transducer array.

17. A method comprising:
generating, one or more acoustic beams directed at a target with one or more of a first transducer array, a second transducer array, and a third transducer array, of an ultrasound system;
receiving, at the second transducer array and the third transducer array, reflected ultrasound that results from reflections of the one or more acoustic beams;
generating imaging data for the fields of view of the second transducer and the third transducer based on the receiving of the reflected ultrasound;
determining, by a computing and imaging device of the ultrasound system, one or more of characteristics of response to one of the one or more acoustic beams and physical changes in the target;
generating, by a computing and imaging device of the ultrasound system, beam adjustments for an acoustic beam generated by the first transducer array based on either or both of determined characteristics of the response to the first acoustic beam and the determined physical changes in the target;
adjusting, by a computing and imaging device, the acoustic beam generated by the first transducer array based on the beam adjustments.

18. The method of claim 16, wherein the second transducer array is arranged at an angle to the third transducer array such that azimuthal data of the second transducer is used to locate the elevational direction of the third transducer array and the azimuthal data of the third transducer is used to locate the elevational direction of the second transducer array.

19. The method of claim 16, wherein the response to the first acoustic beam results in cavitation.

20. The method of claim 16, wherein the determined physical changes in the target comprise one or more of movement of the target and changes to the material of the target.

21. The method of claim 16, further comprising moving the second transducer array and the third transducer array.
